(19) United States
(12) Patent Application Publication
(10) Pub. No.: US 2021/0153886 A1
(43) Pub. Date: May 27, 2021

(54) ENDOSCOPIC GRASPING FORCEPS

(71) Applicant: TOKUSEN KOGYO CO., LTD., Ono-shi, Hyogo (JP)

(72) Inventor: Hiroyuki YAMASHITA, Kobe-shi, Hyogo (JP)

(73) Assignee: TOKUSEN KOGYO CO., LTD., Ono-shi, Hyogo (JP)

(21) Appl. No.: 16/613,455

(22) PCT Filed: May 15, 2018
(86) PCT No.: PCT/JP2018/018811

§ 371 Date: Nov. 14, 2019

(30) Foreign Application Priority Data
Jul. 13, 2017 (JP) 2017-136683

(51) Int. Cl. A61B 17/29; A61B 10/04

(52) U.S. Cl. CPC A61B 17/29; A61B 2017/0034; A61B 10/04

(57) ABSTRACT

Forceps 2 include an insertion tube 6 having a tubular shape, and an operation wire 8 inserted in the insertion tube 6 so as to be movable forward and backward. The operation wire 8 includes a wire body 18, and a plurality of grasping members 20 extending from a leading end of the wire body 18. The grasping members 20 include grasping bodies 26. In an inserted attitude in which the grasping members 20 are inserted in the insertion tube 6, the grasping bodies 26 are arranged in a circumferential direction and a space 24 is formed so as to be surrounded by the grasping bodies 26 arranged in the circumferential direction.

### TECHNICAL FIELD

The present invention relates to endoscopic grasping forceps.

### BACKGROUND ART

JPH8-56951 discloses endoscopic grasping forceps. These forceps are used for examining a body cavity of a patient or performing a surgical operation. These forceps include an insertion tube and an operation wire inserted in the insertion tube. The operation wire includes a wire body and a plurality of grasping members which extend from a leading end of the wire body. The grasping members radially expand upon protrusion from the insertion tube, and radially contract upon insertion into the insertion tube. The grasping members open and close by such radial expansion and contraction. A foreign substance, various cellular tissue specimens including polyps, and the like in a living body can be obtained by the opening and closing of the grasping members. With the use of these forceps, a foreign substance, a cellular tissue specimen, or the like can be collected through a small incision. This consequently reduces the burden on a patient.

### CITATION LIST

### Patent Literature

Patent Literature 1: JPH8-56951

### SUMMARY OF THE INVENTION

### Problems to be Solved by the Invention

Grasping members inserted in an insertion tube are to be inserted into a body cavity together with the insertion tube. The grasping members are to be inserted along a pathway in the body cavity. The pathway is bent, and an external force acts on the insertion tube inserted therein. The external force also acts on the grasping members. The grasping members may be deformed by receiving the external force. The deformed grasping members are likely to interfere with one another. Due to such interference, the grasping members may not be able to radially expand in an orderly manner upon protrusion from the insertion tube. Further, the grasping members may not be able to radially contract in an orderly manner after the radial expansion. Reduced precision in opening and closing of the grasping members will hinder the opening and closing of the grasping members.

The reduced precision in the opening and closing of the grasping members will hinder stable grasping of a foreign substance, a cellular tissue specimen, or the like. A main problem to be addressed by the present invention is to facilitate the opening and closing of the grasping members. In addition to solving the above problem, it is more desirable that stable grasping of a foreign substance, a cellular tissue specimen, or the like is enabled. It is still more desirable that treatment can be given while a foreign substance, a cellular tissue specimen, or the like is stably grasped.

An object of the present invention is to provide endoscopic grasping forceps in which opening and closing of grasping members are facilitated.

### Solution to the Problems

Endoscopic grasping forceps according to the present invention include an insertion tube having a tubular shape, and an operation wire inserted in the insertion tube so as to be movable forward and backward. The operation wire includes a wire body, and a plurality of grasping members extending from a leading end of the wire body. The grasping members include grasping bodies. In an inserted attitude in which the grasping members are inserted in the insertion tube, the grasping bodies are arranged in a circumferential direction. In these forceps, a space is formed so as to be surrounded by the grasping bodies arranged in the circumferential direction.

Preferably, each of the grasping members includes a claw extending radially inward from a leading end of the grasping body.

Preferably, the grasping members are elastically deformed in the inserted attitude. The grasping members are preferably capable of expanding radially outward in a protruded attitude in which the grasping members are protruded from the insertion tube.

Preferably, the grasping members are circumferentially arranged and fixed on an outer circumferential surface of the wire body. On the outer circumferential surface of the wire body, the grasping members circumferentially adjacent to one another are in contact with one another in the circumferential direction.

Preferably, each of the grasping bodies includes a base fixed to the wire body, a leading end, and a large-diameter portion positioned between the base and the leading end. In the protruded attitude, the grasping body preferably extends so as to be slanted radially outward with respect to an axial direction from the base toward the large-diameter portion, and extends so as to be slanted radially inward with respect to the axial direction from the large-diameter portion toward the leading end.

Preferably, a contour of an outer circumferential surface formed by the grasping bodies, in a state of being arranged in contact with one another in the circumferential direction, forms a circular shape in a cross-section perpendicular to an axial direction.

Preferably, in a cross-section of each of the grasping bodies perpendicular to an axial direction, a circumferential width of the grasping body increases from a radially inner side toward a radially outer side.

Preferably, the grasping bodies form a shape of a hollow pipe with circumferential end surfaces of the grasping bodies adjacent to one another being in contact with one another.

Preferably, a cross-section of each of the grasping bodies perpendicular to the axial direction is in a shape of an arc.

Preferably, these forceps include a core member. The wire body is in a shape of a hollow pipe having a through hole. The core member is inserted in the through hole of the wire body and in the space formed by the circumferentially arranged grasping members, in such a manner as to be movable forward and backward in an axial direction.

Preferably, the core member includes a core body and a needle attached to a leading end of the core body.

Preferably, the core body and the needle are hollow.

Preferably, the core member includes a core body and an electrocautery attached to a leading end of the core body.

Preferably, the core member includes a core body and a magnet attached to a leading end of the core body.

### Advantageous Effects of the Invention

In the endoscopic grasping forceps according to the present invention, in the inserted attitude of being inserted in the insertion tube, the grasping bodies are arranged in the circumferential direction so as to form a space on the radially inner side. Accordingly, interference of one of the grasping bodies with a radially opposing one of the grasping bodies is restrained. This restraining of interference prevents the opening and closing of the grasping members from being hindered. In these forceps, the grasping members are able to easily expand radially in the protruded attitude of being protruded from the insertion tube. In these forceps, the grasping members are able to easily open and close.

In these forceps, the core member may be inserted in the through hole of the wire body and the space formed by the grasping members so as to be movable forward and backward. The forceps including an auxiliary grasping tool as a core member enable further stable grasping of a foreign substance, a cellular tissue specimen, or the like with conjunctive use of the auxiliary grasping tool and the grasping members. Further, the forceps including a treatment instrument as a core member enable treatment with use of the treatment instrument while a foreign substance, a cellular tissue specimen, or the like is grasped by the grasping members.

### BRIEF DESCRIPTION OF THE DRAWINGS

FIG. 1 is a conceptual diagram illustrating endoscopic grasping forceps according to an embodiment of the present invention.

FIG. 2 is a cross-sectional view, perpendicular to the axial direction, taken along line II-II in FIG. 1.

FIG. 3 is a cross-sectional view, perpendicular to the axial direction, taken along line III-III in FIG. 1.

FIG. 4 is an explanatory diagram illustrating an inserted attitude of the grasping forceps of the endoscopic grasping forceps illustrated in FIG. 1.

FIG. 5 is an explanatory diagram illustrating a protruding attitude of the grasping forceps of the endoscopic grasping forceps illustrated in FIG. 1.

FIG. 6 is an explanatory diagram illustrating a state where the forceps illustrated in FIG. 1 are used.

FIG. 7(a) is a cross-sectional view illustrating endoscopic grasping forceps according to another embodiment of the present invention, FIG. 7(b) is a cross-sectional view illustrating endoscopic grasping forceps according to still another embodiment of the present invention, and FIG. 7(c) is a cross-sectional view illustrating endoscopic grasping forceps according to yet another embodiment of the present invention.

FIG. 8 is a partial cross-sectional view of endoscopic grasping forceps according to still yet another embodiment of the present invention.

FIG. 9 is a cross-sectional view of a biopsy needle of the forceps illustrated in FIG. 8.

### DESCRIPTION OF EMBODIMENTS

The following will describe in detail the present invention based on preferred embodiments with appropriate reference to the drawings.

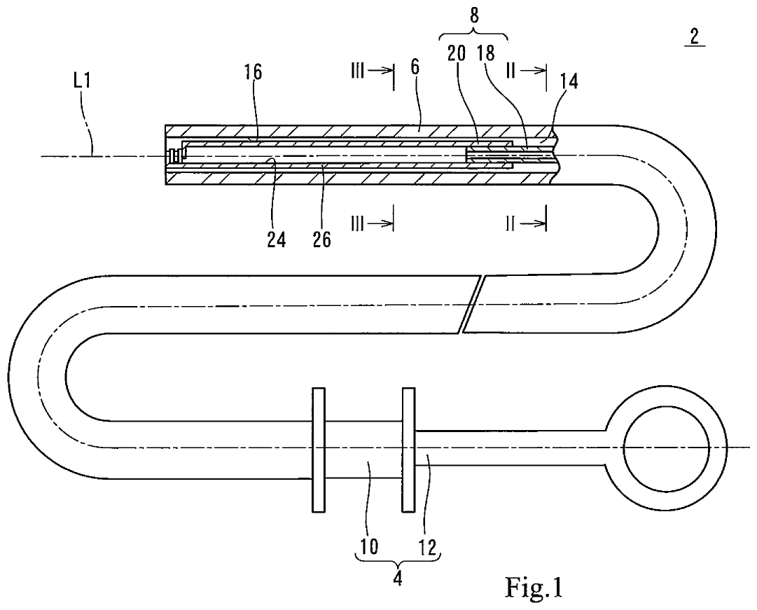

Endoscopic grasping forceps 2 illustrated in FIG. 1 include an operation part 4, an insertion tube 6, and an operation wire 8. An alternate long and short dash line L1 in FIG. 1 represents the axial line of the forceps 2. In the description herein, the direction in which the axial line L1 extends is referred to as an axial direction (also referred to as a front-rear direction), the direction perpendicular to the axial line L1 is referred to as a radial direction, and the direction of rotation about the axial line L1 is referred to as a circumferential direction.

The operation part 4 includes an insertion tube operation part 10 and an operation wire operation part 12. The insertion tube operation part 10 is attached to the insertion tube 6. The operation wire operation part 12 is attached to the operation wire 8.

The insertion tube 6 is in the shape of a hollow pipe. The insertion tube 6 has an inner circumferential surface 16 that surrounds a through hole 14 passing through the insertion tube 6 in the axial direction. The insertion tube 6 has flexibility.

The operation wire 8 includes a wire body 18 and a plurality of grasping members 20. The wire body 18 has flexibility. The operation wire operation part 12 is attached to the rear end of the wire body 18. The operation wire 8 is inserted in the through hole 14 of the insertion tube 6 so as to be movable forward and rearward in the front-rear direction.

The grasping members 20 are fixed to a leading end portion of the wire body 18. The method for this fixing is not particularly limited, but firm fixing is preferable. Examples of the fixing method include welding (laser welding, brazing), crimping of the wire body 18, resin welding, and adhesion using an adhesive.

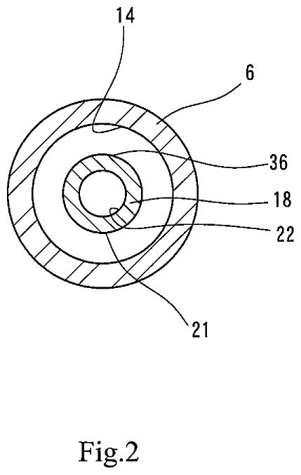

As illustrated in FIG. 2, the wire body 18 is in the shape of a hollow pipe. The contour of the outer circumferential surface 21 of the wire body 18 is circular in the cross-section perpendicular to the axial direction. A wire through hole 22 is formed in the wire body 18. The contour of the inner circumferential surface of the wire body 18 which surrounds the wire through hole 22 is circular in the cross-section perpendicular to the axial direction. The wire through hole 22 extends from the leading end to the rear end of the wire body 18 along the axial line L1 illustrated in FIG. 1. The wire body 18 is, for example, made of a metal, a resin, or a metal coated with a resin. The wire body 18 may be any of those which include the wire through hole 22 and has flexibility, and may be, for example, a hollow rope formed by a plurality of metal wires.

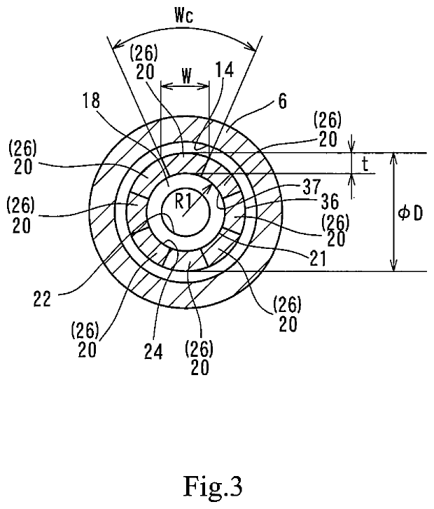

As illustrated in FIG. 3, the grasping members 20 are arranged circumferentially. Each of the grasping members 20 is fixed to the outer circumferential surface 21 of the wire body 18. On the outer circumferential surface 21 of the wire body 18, the grasping members 20 circumferentially adjacent to each other are in contact with each other in the circumferential direction. On the outer circumferential surface 21 of the wire body 18, the grasping members 20 circumferentially adjacent to each other are fixed to each other while being in contact with each other. Examples of the method for this fixing include welding (laser welding, brazing), resin welding, and adhesion using an adhesive. While the grasping members 20 are fixed to the outer circumferential surface 21 of the wire body 18 in this embodiment, the grasping members 20 may be fixed in any manner as long as the grasping members 20 are arranged circumferentially. For example, the grasping members 20 may be fixed to an end surface, of the wire body 18, facing toward the axial direction.

The grasping members 20, arranged circumferentially, form a space 24 on the radially inner side thereof. The grasping members 20 include grasping bodies 26 that surround the space 24. The space 24 is continuous with the wire through hole 22. The forceps 2 include eight grasping members 20, and the number of grasping members 20 is not limited thereto, as long as the number is two or more.

As illustrated in FIG. 3, the contour of the outer circumferential surface 36 formed by the grasping bodies 26 circumferentially arranged on the outer circumferential surface 21 of the wire body 18 is in the shape of a circle. A double-headed arrow ϕD indicates the diameter of this circle. The diameter ϕD is, for example, 3 mm. A double-headed arrow t indicates the thickness of the grasping body 26 in the radial direction. A double-headed arrow W indicates, in the cross-section of FIG. 3, the maximum width of the grasping body 26 in the direction perpendicular to the radial direction. A double-headed arrow Wc indicates, in the cross-section of FIG. 3, the circumferential width of the grasping body 26. The circumferential width Wc is measured along the circumferential direction. The circumferential width Wc gradually increases from the radially inner side toward the radially outer side.

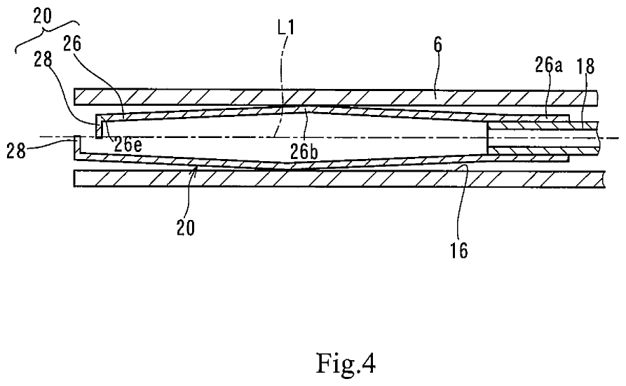

In FIG. 4, the grasping members 20 inserted in the insertion tube 6 are shown. In FIG. 4, the cross-section of two of the grasping members 20 which are radially opposite to each other are shown, while the other grasping members 20 are not shown. Each of the grasping members 20 includes a claw 28 that extends from the leading end of the grasping body 26. The grasping body 26 extends axially forward from a base 26a. The claw 28 extends radially inward from the leading end 26e of the grasping body 26. The grasping body 26 has a large-diameter portion 26b between the base 26a and the leading end 26e. The large-diameter portion 26b is in contact with the inner circumferential surface 16 of the insertion tube 6.

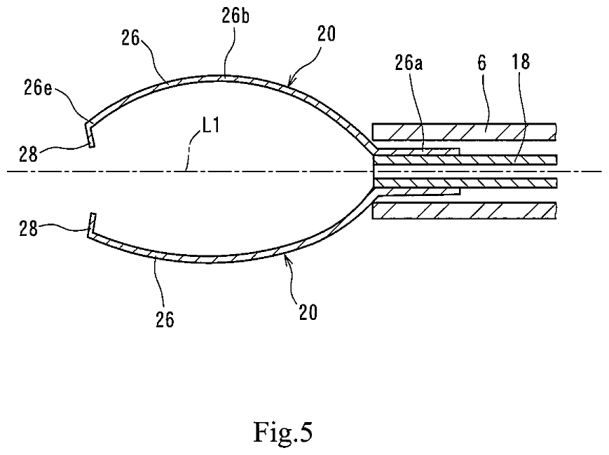

In FIG. 5, the grasping members 20, which have been caused to protrude from the insertion tube 6, are shown. In FIG. 5, the cross-section of the two grasping members 20 are shown, while the other grasping members 20 are not shown. Each of the grasping bodies 26 extends so as to be slanted radially outward with respect to the axial line L1 from the base 26a toward the large-diameter portion 26b. Further, the grasping body 26 extends so as to be slanted radially inward with respect to the axial line L1 from the large-diameter portion 26b toward the leading end 26e. With this position, the large-diameter portion 26b and the leading end 26e are positioned at a radially outer side with respect to the inner circumferential surface 16 of the insertion tube 6 in the radial direction.

In the present invention, the attitude of the grasping members 20 in FIG. 4 is referred to as an inserted attitude. With the inserted attitude, radial expansion of the grasping bodies 26 is restricted by the insertion tube 6. The grasping members 20 are radially contracted. In the inserted attitude, the grasping members 20 are elastically deformed. The attitude of the grasping members 20 in FIG. 5 is referred to as a protruded attitude. With this protruded attitude, the radial restriction by the insertion tube 6 is released. The grasping members 20 are radially expanded. The grasping members 20 have restored the original shapes before the elastic deformation.

In these forceps 2, the operation part 4 is operated to cause the operation wire 8 to move forward and backward with respect to the insertion tube 6. With this forward and backward movement, the grasping members 20 change the attitude between the inserted attitude, which is taken when the grasping members 20 are inserted in the insertion tube 6, and the protruded attitude, which is taken when the grasping members 20 are protruded from the insertion tube 6.

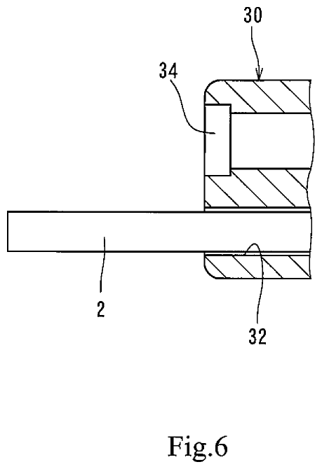

FIG. 6 illustrates a use example of the forceps 2. The forceps 2 are passed through a treatment instrument channel 32 of an endoscope 30. In FIG. 5, the forceps 2 are protruded from the treatment instrument channel 32. A lens 34 of the endoscope 30 is positioned in the vicinity of the treatment instrument channel 32.

The grasping members 20 are brought into the protruded attitude in an observable range of the endoscope 30, which is not shown. A foreign substance, for example, is engaged by the claws 28 of the grasping members 20 brought into the protruded attitude. While the foreign substance is being engaged by the claws 28, the operation part 4 is operated to cause the grasping members 20 to gradually change the attitude from the protruded attitude to the inserted attitude. At this time, the grasping members 20 are together moved radially inward to securely grasp the foreign substance. In this manner, the foreign substance is collected using the forceps 2.

In these forceps 2, the space 24 is formed on the radially inner side so as to be surrounded by the grasping bodies 26. Accordingly, the grasping bodies 26 are restrained from interfering with one another except for those circumferentially adjacent to each other. Interference of one of the grasping bodies 26 with the radially opposing one of the grasping bodies 26 is restrained. Interference of the grasping bodies 26 with one another at the time of opening and closing of the grasping bodies 26 is restrained. The attitude of the grasping members 20 can be changed smoothly between the inserted attitude and the protruded attitude.

In addition, in these forceps 2, the bases 26a of circumferentially adjacent ones of the grasping bodies 26 are fixed to each other. When in the inserted attitude, the grasping bodies 26 are arranged in the circumferential direction. The grasping bodies 26 restrain each other from circumferential deformation. Due to the restraint on deformation, the attitude of the grasping members 20 can be changed smoothly between the inserted attitude and the protruded attitude.

From the viewpoint of restraining the circumferential deformation, it is preferable that the width W of the grasping body 26 is greater than the thickness t thereof.

The grasping members 20 having a higher tensile strength are restrained from deformation more effectively. From this viewpoint, the tensile strength of the grasping members 20 is preferably not less than 2000 MPa, and more preferably not less than 2300 MPa. Meanwhile, the grasping members 20 having an excessively high tensile strength impair the operability of the operation wire in the insertion tube. From the viewpoint of the operability, the tensile strength of the grasping members 20 is preferably not more than 3000 MPa. The tensile strength is measured in accordance with the “JIS Z 2241” standard.

In these forceps 2, the grasping bodies 26 are less likely to interfere with one another even when the number of grasping members 20 is greater than those of conventional ones. By increasing the number of grasping members 20, a foreign substance or the like can be grasped easily. From this viewpoint, the number of grasping members 20 circumferentially arranged is preferably not less than six, more preferably not less than seven, and still more preferably not less than eight. Meanwhile, from the viewpoint of restraining deformation, the grasping members 20 need to have sufficient strength. From this viewpoint, the number of grasping members 20 is preferably not more than 12, more preferably not more than 11, and still more preferably not more than 10.

On the outer circumferential surface 21 of the wire body 18, the grasping bodies 26 are in contact with the circumferentially adjacent ones of the grasping bodies 26. Accordingly, the grasping bodies 26 are restrained from being deformed in the circumferential direction. From the viewpoint of restraining the circumferential deformation, when being in the inserted attitude, the grasping bodies 26 circumferentially adjacent to each other are preferably in contact with each other in a range from the leading end of the wire body 18 to the leading ends 26e of the grasping bodies 26. For example, the grasping bodies 26 circumferentially adjacent to each other are preferably in contact with each other at or near the leading ends 26e of the grasping bodies 26. This contact further reduces the likelihood of a circumferential deformation of the grasping members 20. Accordingly, the attitude of the grasping members 20 can be changed further smoothly between the inserted attitude and the protruded attitude.

The claw 28 of each of the grasping members 20 extends radially inward from the leading end 26e of the corresponding grasping body 26. The claw 28 can be easily made by bending radially inward the leading end portion of a material that forms the grasping body 26.

When being in the inserted attitude, the grasping members 20 are inserted in the insertion tube 6. Thus, the grasping members 20 are able to smoothly move in a body cavity. When being in the protruded attitude, each of the grasping members 20 expands radially outward. A foreign substance or the like in the body can be easily collected by changing the attitude between the protruded attitude and the inserted attitude.

When being in the protruded attitude, each of the grasping bodies 26 extends so as to be slanted radially outward with respect to the axial direction from the base 26a toward the large-diameter portion 26b. Further, the grasping body 26 extends so as to be slanted radially inward with respect to the axial direction from the large-diameter portion 26b toward the leading end 26e. Since the grasping body 26 extends so as to be slanted radially inward from the large-diameter portion 26b toward the leading end 26e, grasping of a foreign substance by the claws 28 is facilitated.

In addition, since the one in contact with the inner circumferential surface 16 of the insertion tube 6 is not the leading end 26e but the large-diameter portion 26b, the operation wire 8 is able to easily move forward and backward, and thus is able to easily rotate about the rotation axis extending in the axial direction. In these forceps 2, the radius of curvature of the outer circumferential surface 36 formed by the grasping bodies 26 is smaller than the radius of curvature of the inner circumferential surface 16 of the insertion tube 6. Accordingly, the operation wire 8 can be rotated more easily.

In these forceps 2, the outer circumferential surface 36 formed by the grasping bodies 26, in a state of being arranged in contact with one another in the circumferential direction, has a circular shape in the cross-section perpendicular to the axial direction. The operation wire 8 is able to easily rotate, with respect to the insertion tube 6, about the axial line L1 as the rotation axis. These forceps 2 have excellent operability of the operation wire 8.

In the cross-section illustrated in FIG. 3, the circumferential width We of each of the grasping bodies 26 increases from the radially inner side toward the radially outer side. This enables the circumferentially adjacent grasping members 20 to restrain each other from being deformed radially inward. The grasping members 20 are restrained from being deformed by an external force. From the viewpoint of restraining this deformation, the cross-section of the grasping members 20 (grasping bodies 26) is preferably formed in the shape of a hollow pipe, with circumferential end surfaces of the grasping bodies 26 adjacent to each other being in contact with each other, as illustrated in FIG. 3. It is preferable that, in this cross-section, the contour of a portion of the outer circumferential surface 36 which corresponds to each of the grasping members 20 is in the shape of an arc, and that the contour of a portion of an inner circumferential surface 37 surrounding the space 24 is also in the shape of an arc. The radius of curvature R1 of the contour of the inner circumferential surface 37 is preferably set to be equal to the radius of curvature of the outer circumferential surface 21 of the wire body 18. From the viewpoint of facilitating production of the grasping members 20, the cross-section of each of the grasping bodies 26 is preferably in the shape of an arc. In the arc-shaped cross-section, the middle position of the arc-shape in the portion of the contour of the outer circumferential surface 36 falls in line with the middle position of the arc-shape in the portion of the contour of the inner circumferential surface 37. In addition, it is preferable that the contour of the circumferential end surfaces of each of the grasping bodies 26, which are in contact with the circumferential end surfaces of the grasping bodies 26 that are circumferentially adjacent to the grasping body 26, extend in the radial direction.

It should be noted that, in the present invention, the cross-section of each of the grasping bodies 26 may not necessarily be shaped such that the corresponding portion of the outer circumferential surface 36 and the corresponding portion of the inner circumferential surface 37 are each in the shape of an arc as illustrated in FIG. 3. The cross-section of the grasping body 26 may be such that corners of the shape formed from the arc-shaped portions of the outer circumferential surface 36 and the inner circumferential surface 37 as illustrated in FIG. 3 are chamfered, for example, R-chamfered or C-chamfered. It should also be noted that the cross-section of the grasping body 26 may be, for example, in the shape of a rectangle. The rectangle may include those obtained by chamfering, for example, R-chamfering or C-chamfering, the corners. It should also be noted that the cross-section of the grasping body 26 may be in the shape of a crescent or an oval track, and that such a cross-section may include those obtained by chamfering, for example, R-chamfering or C-chamfering, the corners.

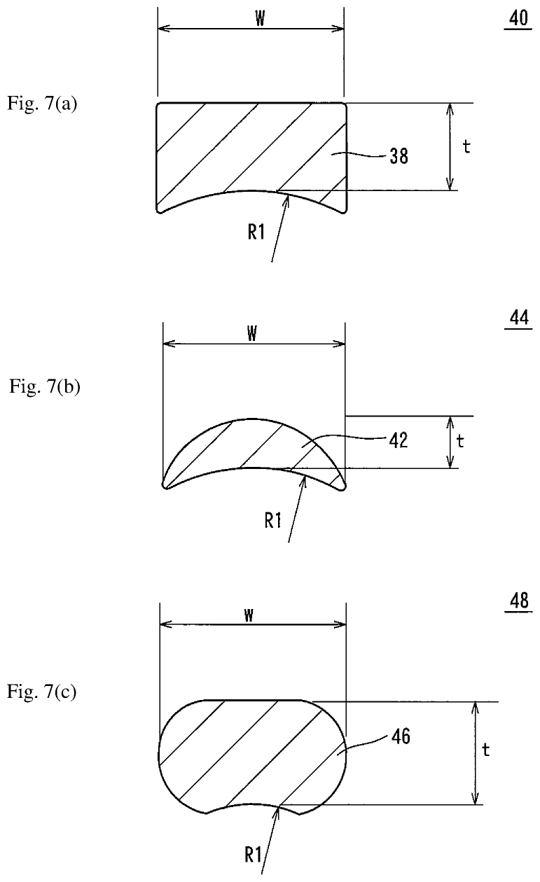

As illustrated in FIG. 7(a), the cross-section of a grasping body 38 may be in a shape approximate to a rectangle and have an arc-shaped radially inner contour. These forceps 40 are similar to the forceps 2 except that the cross-sectional shape of the grasping body 26 is replaced with the cross-sectional shape of the grasping body 38.

In these forceps 40, the radially inner contour, in the cross-section, of the grasping body 38 is formed in the shape of an arc having a radius of curvature R1. Thus, a plurality of the grasping bodies 38 can be easily arranged circumferentially along the outer circumferential surface 21 of the wire body 18. From this viewpoint, the radius of curvature R1 is preferably set to be equal to the radius of curvature of the outer circumferential surface 21 of the wire body 18. The width W is preferably greater than the thickness t.

Further, as illustrated in FIG. 7(b), a cross-section of a grasping body 42 may be in the shape of a crescent. These forceps 44 are similar to the forceps 2 except that the cross-sectional shape of the grasping body 26 of the forceps 2 is replaced with the cross-sectional shape of the grasping body 42. In the cross-section of the grasping body 42 as well, the radius of curvature R1 of the radially inner arc is preferably set to be equal to the radius of curvature of the outer circumferential surface 21 of the wire body 18. The width W is preferably greater than the thickness t.

Further, as illustrated in FIG. 7(c), the cross-section of a grasping body 46 may be in a shape approximate to an oval track and have an arc-shaped radially inner contour. These forceps 48 are similar to the forceps 2 except that the cross-sectional shape of the grasping body 26 of the forceps 2 is replaced with the cross-sectional shape of the grasping body 46. In these forceps 48, a radially inner contour, in the cross-section, of the grasping body 46 is formed in the shape of an arc having a radius of curvature R1. The radius of curvature R1 of this arc is preferably set to be equal to the radius of curvature of the outer circumferential surface 21 of the wire body 18. The width W is preferably greater than the thickness t.

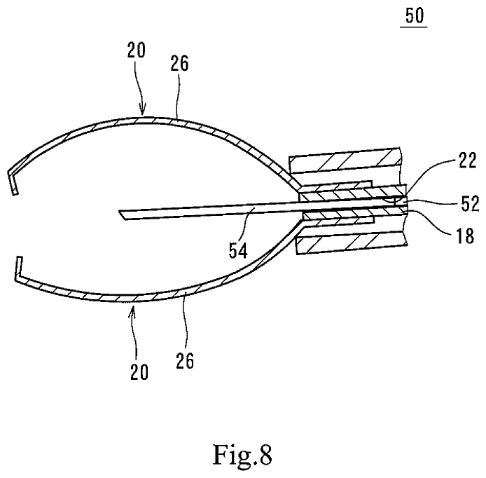

FIG. 8 illustrates a part of endoscopic grasping forceps 50 according to still yet another embodiment of the present invention. Components of the forceps 50 which are different from those of the forceps 2 will be described below, whereas components of the forceps 50 which are the same as those of the forceps 2 are omitted. Components of the forceps 50 which are the same as those of the forceps 2 will be described using the same reference numerals.

These forceps 50 have, as a core member, a core body 52 and a biopsy needle 54. The core body 52 and the biopsy needle 54 are inserted in the wire through hole 22. The core body 52 is formed by a flexible tube. The core body 52 extends from the rear end of the biopsy needle 54. The core body 52 and the biopsy needle 54 are inserted in a wire body 18 so as to be movable forward and backward in the front-rear direction. The operation part 4 includes a biopsy needle operation part, which is not shown. The core body 52 and the biopsy needle 54 can be caused to move forward and backward with respect to grasping members 20 by operating the biopsy needle operation part.

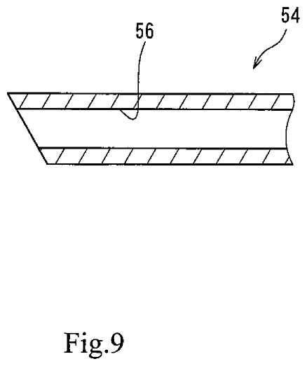

As illustrated in FIG. 9, the biopsy needle 54 has a needle through hole 56 formed therein. The core body 52 also has formed therein a through hole continuous to the needle through hole 56, which is not shown.

In these forceps 50 as well, a plurality of the grasping bodies 26 are arranged in the circumferential direction when being in the inserted attitude, and this restrains circumferential deformation thereof. Interference of the grasping bodies 26 with one another in the radial direction is restrained. Since the grasping bodies 26 are less likely to interfere with one another, the number of grasping members 20 may be greater than those of conventional ones. By increasing the number of grasping members 20, a foreign substance or the like can be grasped further easily.

Since the space 24 is formed in the forceps 50, interference of the biopsy needle 54 with the grasping members 20 is restrained. The biopsy needle 54 is easily movable forward and backward.

In these forceps 50, the number of grasping members 20 may be greater than those of conventional ones. By increasing the number of grasping members 20, even a small tumor or the like can be easily grasped at a fixed position. Even a tumor or the like smaller than 1 cm can be easily pricked by the biopsy needle 54. With use of these forceps 50, a tumor having a size of 1 mm or larger can be pricked by the biopsy needle 54. These forceps 50 allow even a small tumor to be grasped by the grasping members 20 with conjunctive use of the biopsy needle 54 serving as an auxiliary grasping tool. From the viewpoint of stable grasping, the number of grasping members 20 in the forceps 50 is preferably not less than six, more preferably not less than seven, and still more preferably not less than eight.

In these forceps 50, each of the biopsy needle 54 and the core body 52 is hollow. A sample, such as a cellular tissue specimen, can be collected using the biopsy needle 54. In addition, a chemical agent can be injected through the core body 52 and the biopsy needle 54. These forceps 50 can be used to collect a sample or inject a chemical agent through a small incision. These forceps 50 include the biopsy needle 54 as a treatment instrument, and thus treatment can be given on a tumor or the like while being grasped using the biopsy needle 54.

While the biopsy needle 54 has been described by way of example herein, such example is not limited to the biopsy needle. For example, the core member may include the core body 52 and an electrocautery attached to the leading end of the core body 52. The electrocautery may be used as a treatment instrument. Alternatively, the core member may include the core body 52 and a magnet attached to the core body 52. The magnet may be used as an auxiliary grasping tool. The grasping members and the magnet can be used to securely grasp and extract a magnetic foreign substance or the like in a body.

### INDUSTRIAL APPLICABILITY

The forceps described above may be widely used as endoscopic grasping forceps to be inserted into not only human bodies but also animal bodies.

### DESCRIPTION OF THE REFERENCE CHARACTERS

2, 40, 44, 48, 50 . . . forceps
6 . . . insertion tube
8 . . . operation wire
14 . . . through hole
16 . . . inner circumferential surface
18 . . . wire body
20 . . . grasping member
24 . . . space
26, 38, 42, 46 . . . grasping body
26a . . . base
26b . . . large-diameter portion
26e . . . leading end
28 . . . claw
36 . . . outer circumferential surface
52 . . . core body
54 . . . biopsy needle

1. Endoscopic grasping forceps comprising an insertion tube having a tubular shape, and an operation wire inserted in the insertion tube so as to be movable forward and backward, wherein
the operation wire includes a wire body, and a plurality of grasping members extending from a leading end of the wire body,
the grasping members include grasping bodies, and
in an inserted attitude in which the grasping members are inserted in the insertion tube, the grasping bodies are arranged in a circumferential direction and a space is formed so as to be surrounded by the grasping bodies arranged in the circumferential direction.

2. The forceps according to claim 1, wherein each of the grasping members includes a claw extending radially inward from a leading end of the grasping body.

3. The forceps according to claim 1, wherein the grasping members are elastically deformed in the inserted attitude, whereas the grasping members are capable of expanding radially outward in a protruded attitude in which the grasping members are protruded from the insertion tube.

4. The forceps according to claim 1, wherein the grasping members are circumferentially arranged and fixed on an outer circumferential surface of the wire body, and on the outer circumferential surface of the wire body, the grasping members circumferentially adjacent to one another are in contact with one another in the circumferential direction.

5. The forceps according to claim 1, wherein
each of the grasping bodies includes a base fixed to the wire body, a leading end, and a large-diameter portion positioned between the base and the leading end; and
in the protruded attitude, the grasping body extends so as to be slanted radially outward with respect to an axial direction from the base toward the large-diameter portion, and extends so as to be slanted radially inward with respect to the axial direction from the large-diameter portion toward the leading end.

6. The forceps according to claim 1, wherein a contour of an outer circumferential surface formed by the grasping bodies, in a state of being arranged in contact with one another in the circumferential direction, forms a circular shape in a cross-section perpendicular to an axial direction.

7. The forceps according to claim 1, wherein, in a cross-section of each of the grasping bodies perpendicular to an axial direction, a circumferential width of the grasping body increases from a radially inner side toward a radially outer side.

8. The forceps according to claim 7, wherein the grasping bodies form a shape of a hollow pipe with circumferential end surfaces of the grasping bodies adjacent to one another being in contact with one another.

9. The forceps according to claim 6, wherein a cross-section of each of the grasping bodies perpendicular to the axial direction is in a shape of an arc.

10. The forceps according to claim 1, comprising a core member, wherein
the wire body is in a shape of a hollow pipe having a through hole; and
the core member is inserted in the through hole of the wire body and in the space formed by the circumferentially arranged grasping members, in such a manner so as to be movable forward and backward in an axial direction.

11. The forceps according to claim 10, wherein the core member includes a core body and a needle attached to a leading end of the core body.

12. The forceps according to claim 11, wherein the core body and the needle are hollow.

13. The forceps according to claim 10, wherein the core member includes a core body and an electrocautery attached to a leading end of the core body.

14. The forceps according to claim 10, wherein the core member includes a core body and a magnet attached to a leading end of the core body.
